(19) United States
(12) Patent Application Publication
(10) Pub. No.: US 2012/0277563 A1
(43) Pub. Date: Nov. 1, 2012

(54) GLUCOSE MONITORING METHOD AND DEVICE FOR TREATING HUMANS

(76) Inventor: Alan G. Balu, Hockessin, DE (US)

(21) Appl. No.: 13/456,905

(22) Filed: Apr. 26, 2012

Related U.S. Application Data
(60) Provisional application No. 61/479,600, filed on Apr. 27, 2011.

(51) Int. Cl. A61M 5/168; A61B 5/145

(52) U.S. Cl. 600/365; 604/891.1

(57) ABSTRACT

This invention relates to a monitoring and/or stabilizing glucose level in the blood stream in a human body signaling the person in the event glucose is at a dangerous level, said method comprising the following steps:
(A) implanting a device in the human body wherein a tube from said device is inserted into a blood vessel and in the blood stream of a person;
(B) continuously monitoring said glucose level in the blood stream with said implanted device, at a set frequency of time;
(C) signaling said person in the event the glucose level drops below a specific level;
(D) optionally injecting, at least once, a specified dose of glucagon from a glucagon reservoir, into the subcutaneous tissue of said person;
(E) alerting medical providers when the glucose level remains below said specific level for at least two consecutive measurements of said glucose level in the blood stream; provided said signal is not manually silenced by the person.

### FIELD OF INVENTION

This invention relates to a method and device for monitoring and/or stabilizing glucose level in the blood stream in a body of a human, signaling the person in the event glucose is at a dangerous level, providing glucagon to the person and notifying medical providers in the event the glucose remains at a dangerous level.

### BACKGROUND

Older patients with diabetes often are limited in their ability to manage diabetes factors because of poor vision, arthritis, dementia, and inability to call for help or dial 911. These patients also have problems in recognizing hypoglycemic episodes making them vulnerable to complications of hypoglycemia. This is also true with pediatric patients. The most severe hypoglycemic events in recent Diabetes Control and Complications Trial occurred during sleep hours. Similarly, it was found that 75% of hypoglycemic seizures in children occurred during sleep. It has also been reported that among patients aged less than 40 years who died over a 10-year period, 6% of the deaths were due to “dead-in-bed” syndrome, which in many instances was probably the result of severe nocturnal hypoglycemia.

Typically, a treatment for hypoglycemia for a diabetic patient is an injection of glucagon. Glucagon is an antagonist to insulin. Paramedics will often treat a patient that is unable to eat and/or is unconscious, with an injection of glucagon. Glucagon, a hormone, raises the blood sugar level naturally and is safe.

It would be desirable to have a method and device that monitors a patient's glucose level in the blood stream, signals the patient when the glucose level drops below a level considered dangerous (generally accepted level is 50), and in the event the patient does not respond, injects the patient with glucagon and notifies medical authorities (for example, by dialing 911) to come to the aid of the patient. The present invention provides such method and device.

### SUMMARY OF INVENTION

This invention relates to a method for monitoring and/or stabilizing glucose level in the blood stream in a human body signaling the person in the event glucose is at a dangerous level, said method comprising the following steps:
(A) implanting a device in the human body wherein a tube from said device is inserted into a blood vessel and in the blood stream of a person;
(B) continuously monitoring said glucose level in the blood stream with said implanted device, at a set frequency of time;
(C) signaling said person in the event the glucose level drops below a specific level;
(D) optionally injecting, at least once, a specified dose of glucagon from a glucagon reservoir, into the subcutaneous tissue of said person;
(E) alerting medical providers when the glucose level remains below said specific level for at least two consecutive measurements of said glucose level in the blood stream; provided said signal is not manually silenced by the person.

This invention also relates to a device for monitoring and/or stabilizing glucose level in the blood stream in a human body, wherein said device can be implanted into said human body, comprising:
(A) an enclosure capable of being implanted into said human body;
(B) a means attached to said enclosure for insertion into a blood vessel for monitoring said glucose level in the blood stream;
(C) a glucagon reservoir positioned in the enclosure, wherein said glucagon is injected into the blood stream in the event said glucose level drops below a specific level;
(D) an injector means connected to said enclosure for said glucagon;
(E) a computer means positioned in said enclosure that records information comprising said glucose level and the time the level is determined, wherein said computer means sends such information to a read-out means worn by a person, signaling said person when said glucose level is below a specific level, and in the event said level remains below said specific level, signaling medical authority, and optionally being connected to the injector means capable of actuating to inject a dose of glucagon into the blood stream.

### BRIEF DESCRIPTION OF THE DRAWINGS

Embodiments of the present invention will be described with reference to the accompanying drawings, wherein like reference numerals denote like parts, and in which:

FIG. 1 illustrates the front cross section of the device implanted in a human body;

FIG. 2 illustrates the back cross section of the device implanted in a human body;

FIG. 3 shows the device implanted under the skin of a human body;

FIG. 4 shows the watch like receiving device worn by a person having an implant of the device; and

FIG. 5 illustrates the opened watch like device and the information received by the device.

### DETAILED DESCRIPTION OF THE INVENTION

Typically, the novel device of this invention is inserted under the skin of a person. Under some circumstances the device can be worn externally but there must be a means to monitor the blood of the patient. For example, a catheter can be inserted into a blood vessel and connected to the external device.

The device of the invention comprises two parts: (1) a monitor comprising a glucagon reservoir, under the skin, with a built-in chip to program the device; and (2) a flip-watch that acts as an alarm, which the patient wears at all times. The device comprises a catheter that stays in the blood stream and continuously monitors blood sugar (glucose), for example, every 15 minutes. The blood sugar can be monitored by minutes listed in the following paragraph:

1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100.

In one embodiment, if the blood sugar is less than 50 units (generally, mg/dL), as is known to a skilled person in the art, it sends an electronic signal to the flip-watch the patient is wearing. It alerts the patient with an alarm sound and vibration to eat something and take care of his low blood sugar. If the blood sugar drops again for the next reading, which, for example, is 15 minutes later, and if the patient does not manually silence the second alarm, the device alerts authorities (911 or a help line) just like any other medical alert devices with an automatic alert system. This will automatically call the preprogrammed help line, or 911, assuming that the patient is not alert enough to eat. Meanwhile, in an optional step, a small dose of glucagon residing in a reservoir of the device is pumped and released into the subcutaneous tissue to elevate the critical blood sugar level of the patient. In one embodiment, the device comprises an electronic chip that can be accessed with specialized computer program to obtain a print-out of the blood sugar level and the number of times glucagon was released from the reservoir over a period of time. The pump is refillable when glucagon is used up. A septum on top of the reservoir is accessible with a syringe to refill glucagon. The septum is one-way septum so that the glucagon does not leak out into the body. A physician can determine the glucagon level in the reservoir by accessing the micro-chip that is built into the device inside the body. The physician can synchronize the device to obtain blood sugar readings, can calibrate the machine, and also program the device to release required amount of glucagon according to the patient's age. This device provides physicians a the person's blood sugar pattern, the time of the day that hypoglycemia happens, the number of times glucagon was injected into the blood stream, and the amount of glucagon left in the reservoir. In one embodiment, the flip-watch has an in-built alarm that beeps differently if the glucagon level in the reservoir is too low.

The alarm can be programmed to provide an electronic signal when the blood sugar level drops below any one of the following set of numbers:

25, 26, 27, 28, 29,30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110.

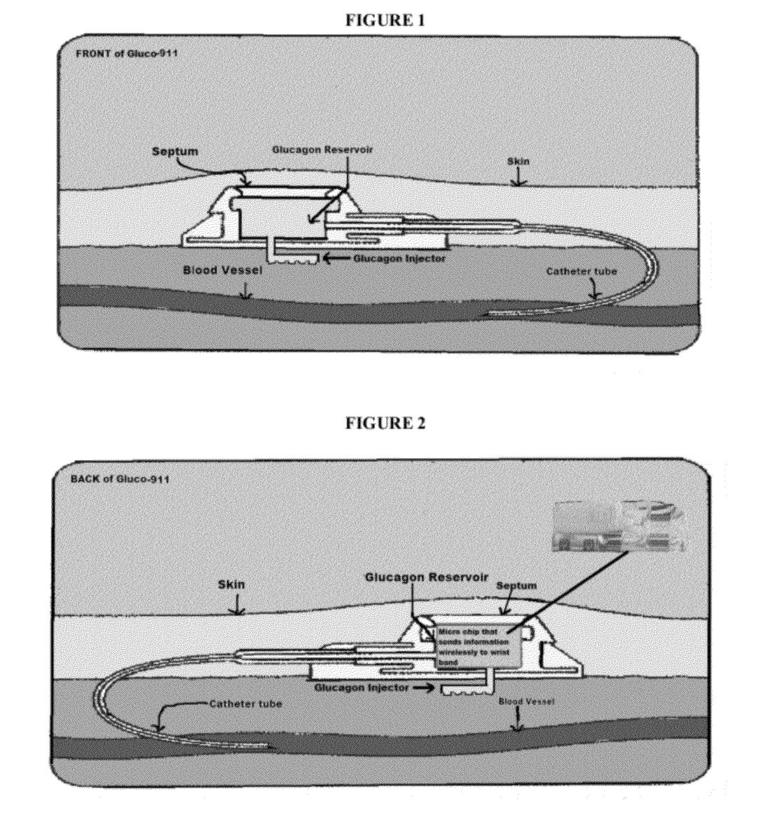

FIG. 1 shows a front cross section of the device implanted in a human body. Typically, the device is implanted below the skin. Basically, the device comprises an enclosure capable of withstanding body fluid and which can be implanted in the body. A tubular means, typically, a catheter, is inserted into a blood vessel to monitor the glucose content of the blood in conjunction with the device usually every 15-minute period. A reservoir for glucagon is positioned in the enclosure connected to a glucagon injector that injects glucagon, when activated by the device, into the subcutaneous tissue to increase the glucose level of the blood. The reservoir is covered by a septum to allow for replenishment of the glucagon that is accomplished with a syringe. The septum is a one way septum that does not allow glucagon to leak into the body.

FIG. 2 shows a back-side cross section of the device, wherein an embedded micro-chip sends information to a receiving device, typically, a wrist band having a watch-like attachment that shows the glucose reading as well as the time when it was taken.

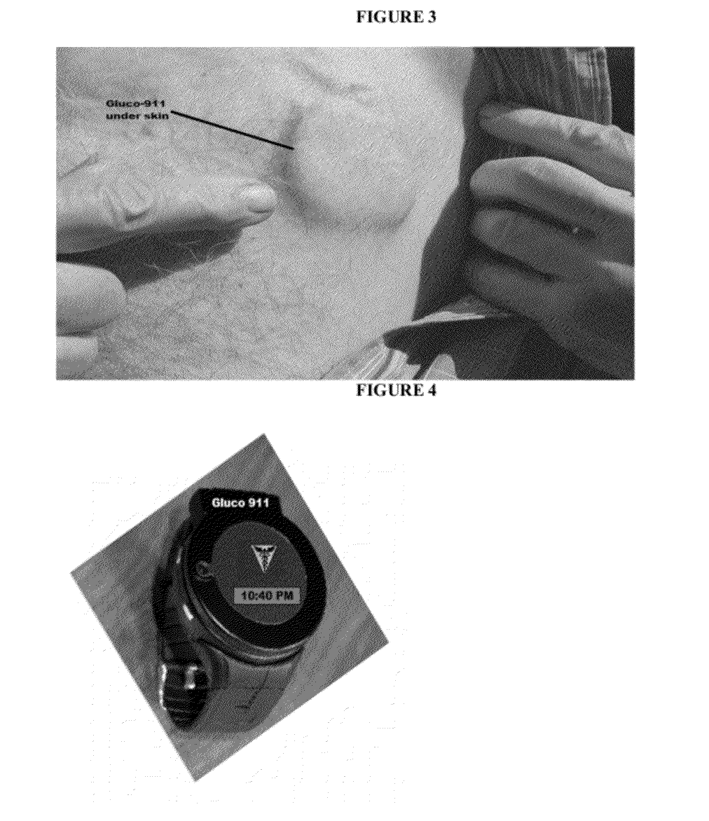

FIG. 3 shows the device implanted under the skin of a person and appears as a small bump on the skin and is not very noticeable.

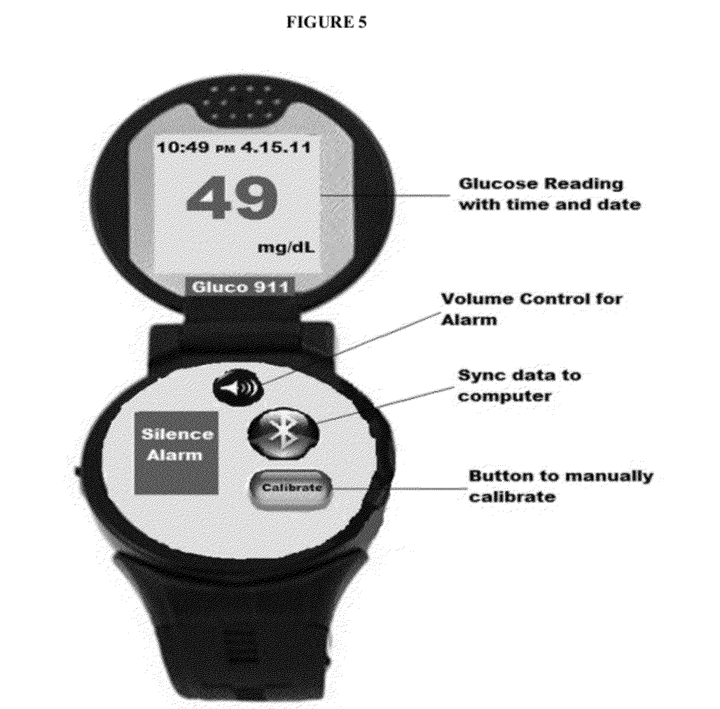

FIG. 4 shows a receiving device that usually is worn like a watch on the wrist. This receiving device can be kept in a pocket or purse but it is preferred to have a person wear the device for easy accessibility, and in the event a signal is transmitted the signal can easily be heard, seen, or felt by the person.

FIG. 5 shows the receiving device with its cover open, showing the time and date of the glucose reading, a volume control for the alarm, a place to silence the alarm, a synchronizing control to synchronize the transmission of data to a computer, and a manual transmission button.

While this invention has been described with reference to illustrative embodiments and examples, the description is not intended to be construed in a limiting sense. Various modifications of the illustrative embodiments, as well as other embodiments of the invention, will be apparent to persons skilled in the art upon reference to this description. For example, as will be apparent to persons skilled in the art, while a number of parts are described as being present in the singular or as a pair, there could be one, two or more of these components present in the apparatus of the present invention. Further, the present invention also encompasses a system for performing the process of the present invention. As will be apparent to a person skilled in the art, while the invention has been described in terms of a single apparatus, the various steps of the process could be performed by different apparatuses that form part of a larger system.

1. A method for monitoring and/or stabilizing glucose level in the blood stream in a human body signaling the person in the event glucose is at a dangerous level, said method comprising the following steps:
(A) implanting a device in the human body wherein a tube from said device is inserted into a blood vessel and in the blood stream of a person;
(B) continuously monitoring said glucose level in the blood stream with said implanted device, at a set frequency of time;
(C) signaling said person in the event the glucose level drops below a specific level;
(D) optionally injecting, at least once, a specified dose of glucagon from a glucagon reservoir, into the subcutaneous tissue of said person;
(E) alerting medical providers when the glucose level remains below said specific level for at least two consecutive measurements of said glucose level in the blood stream; provided said signal is not manually silenced by the person.

2. The method as recited in claim 1, wherein said specific level of glucose is 50.

3. The method as recited in claim 1, wherein said person whose glucose level being monitored has a receiving device that comprises a display for the glucose level and the time when the glucose level was measured, and an alarm mechanism to alert said person if the glucose level falls below a specified level.

4. The method as recited in claim 1, wherein the device contains a computer means that records information comprising the glucose level and the time the level is determined; sends such information to a read-out means worn by the person; signals the person when the glucose level is below a specific level, and in the event the level remains below said specific level, optionally actuates the device to inject a dose of glucagon; and sends a signal to a medical authority.

5. The method as recited in claim 4, wherein the computer means comprises a computer chip and records in the computer chip are accessible and can be downloaded via a computer program.

6. The method as recited in claim 1, wherein said glucagon reservoir is covered by a septum wherein glucagon is replenished by an injection of glucagon through said septum when required.

7. The method as recited in claim 1, wherein said septum is a one-way septum.

8. A device for monitoring and/or stabilizing glucose level in the blood stream in a human body, wherein said device can be implanted into said human body, comprising:
(A) an enclosure capable of being implanted into said human body;
(B) a means attached to said enclosure for insertion into a blood vessel for monitoring said glucose level in the blood stream;
(C) a glucagon reservoir positioned in the enclosure, wherein said glucagon is injected into the blood stream in the event said glucose level drops below a specific level;
(D) an injector means connected to said enclosure for said glucagon;
(E) a computer means positioned in said enclosure that records information comprising said glucose level and the time the level is determined, wherein said computer means sends such information to a read-out means worn by a person, signaling said person when said glucose level is below a specific level, and in the event said level remains below said specific level, signaling medical authority, and optionally being connected to the injector means capable of actuating to inject a dose of glucagon into the blood stream.

9. The device as recited in claim 8, wherein said specific level of glucose is 50.

10. The device as recited in claim 8, further comprising a septum covering said reservoir, whereby said glucagon in said reservoir is replenished by injection of additional glucagon through said septum.

11. A read-out means, worn by said person in conjunction with said device as recited in claim 8, that records the glucose levels and the time the levels were determined and that signals said person when said glucose level is below a specific level.
